(19) United States
(12) Patent Application Publication
(10) Pub. No.: US 2019/0282550 A1
(43) Pub. Date: Sep. 19, 2019

(54) METHOD OF TREATMENT

(71) Applicant: The University of Melbourne, Melbourne, Victoria (AU)

(72) Inventors: Stephen Tong, Melbourne, Victoria (AU); Tu'uhevaha Lino, Melbourne, Victoria (AU); Roxanne Hastie, Melbourne, Victoria (AU)

(21) Appl. No.: 16/349,199

(22) PCT Filed: Nov. 17, 2016
(86) PCT No.: PCT/AU2016/051075

§ 371 Date: May 10, 2019

(51) Int. Cl. A61K 31/439; A61K 31/5377; A61K 9/00; A61P 15/08

(52) U.S. Cl. CPC A61K 31/439; A61P 15/08; A61K 9/0053; A61K 31/5377

(57) ABSTRACT

A therapeutic protocol to induce placenta cell death when required in the treatment of an adverse gynecological condition is applicable, inter alia, to the treatment of ectopic pregnancy, including unruptured ectopic pregnancy and placenta tumors, such as a hydatidiform molar pregnancy or choriocarcinoma. The protocol includes administering vinorelbine or a pharmacologically active and pharmaceutically acceptable salt, solvate, tautomer, stereoisomer, derivative, biosimilar or prodrug form of vinorelbin in an amount effective to induce placenta cell death

### FIELD

The present invention relates generally to a therapeutic protocol to induce placenta cell death when required in the treatment of an adverse gynecological condition. The therapeutic protocol is applicable inter alia to the treatment of ectopic pregnancy including unruptured ectopic pregnancy and placental tumors such as a hydatidiform molar pregnancy or choriocarcinoma.

### BACKGROUND

Bibliographic details of the publications referred to by author in this specification are collected alphabetically at the end of the description.

Reference in this specification to any prior publication (or information derived from it), or to any matter which is known, is not, and should not be taken as an acknowledgement or admission or any form of suggestion that the prior publication (or information derived from it) or known matter forms part of the common general knowledge in the field of endeavor to which this specification relates.

Gynecological disorders create an enormous burden on the health and wellbeing of pregnant mothers and families and communities as a whole. It also has the potential for significant commercial loss to the non-human animal husbandry industry. In either case, the potential economic and clinical care burden to the human and animal health systems is quite significant.

Of particular concern is the development of a gynecological condition where the target tissue for treatment is the placenta. Examples include an ectopic pregnancy and gestational trophoblastic disease of the placenta. An ectopic pregnancy occurs when the conceptus implants outside the uterus (extrauterine pregnancy), generally in the Fallopian tube (Kirk et al. (2014) Hum Reprod Update 20(2):250-261) although extratubular ectopics can occur in the abdomen, cervix, ovaries, the uterine cornua (interstitial ectopic pregnancy) and a previous cesarean scar. An ectopic pregnancy is life threatening as it can erode into maternal vasculature and cause fatal bleeding. Ectopic pregnancy is relatively common, accounting for 1-2% of all pregnancies (Farquhar (2005) Lancet 366 (9485):583-591; Kirk et al. (2014) supra) or 5,000 cases/year in Australia and 100,000 cases/year in the United States of America (Anon (1995) Morbidity Mortality Weekly Report 44:46-48) and 10,000 cases/year in the United Kingdom.

It is notable that an ectopic pregnancy is a potential threat to a mother's life and is the most common cause of death during the first trimester (Cecchno et al. (2014) Arch Glynecol Obstet 290(3):417-423). Hence, ectopic pregnancy is a significant gynecological disease.

Currently, most ectopic pregnancies are surgically excised requiring an anesthetic. An alternative pharmacological treatment is intramuscular methotrexate. However, this treatment is generally only effective for smaller ectopic pregnancies that fulfil strict clinical criteria such as serum human chorionic gonadotropin (hCG) levels of <3000 IU/L, no fetal cardiac activity on ultrasound and a gestational sac size of <3 cm (Mol et al. (2008) Hum Reprod Update 14(4):309-319; Anon (2008) Obstet Gynecol 111(6):1479-1485). If an ectopic pregnancy falls outside these parameters then the rates of failure become unacceptably high. Hence, only a minority of ectopic pregnancies are suitable for methotrexate treatment (Farquar et al. (2005) supra).

Another condition that can be associated with the placenta is the presence of molar pregnancy (hydatidiform mole; a placental tumor that is locally erosive within the uterus) or choriocarcinoma, a malignant gestational trophoblastic cancer. The characterizing feature is intimately related syncytiotrophoblasts and cytotrophoblasts. Choriocarcinoma of the placenta can be preceded by a hydatidiform molar pregnancy or a spontaneous miscarriage. Methotrexate or hysterectomy are common forms of treatment. Sometimes after hysterectomy for either hydatidiform molar pregnancy or choriocarcinoma there is persistent disease that can be resistant to follow-up medical therapy with drugs such as methotrexate. Therefore, new efficacious medical treatments are required for ectopic pregnancies, hydatidiform molar pregnancies and choriocarcinomas.

### SUMMARY

The present invention provides a therapeutic protocol that targets placenta cells to treat an adverse gynecological condition. The protocol enables efficient induction of placenta cell death including activation of apoptosis whilst not being harmful to the Fallopian tube or subsequent fertility. Such a protocol is useful in the treatment of an ectopic pregnancy, generally but not exclusively stable, unruptured ectopic pregnancy and the treatment of a hydatidiform molar pregnancy and a choriocarcinoma of the placenta or other adverse gynecological condition associated with the placenta such as a placental tumor. The protocol comprises the administration of vinorelbine or a pharmacologically active and pharmaceutically acceptable salt, solvate, tautomer, stereoisomer, derivative, biosimilar or prodrug form thereof. The present therapeutic protocol enables an alternative to surgery or the use of methotrexate alone.

The vinorelbine may be administered alone or in combination with another active agent such as an epidermal growth factor receptor (EGFR) inhibitor (e.g. gefitinib, erlotinib/tarceva, certuximab, lapatinib/tykerb, panitumumab/vectibix, theraCIMh-R3/nimotuzumab, matuzumab, MDX447, PKI166, Cl-1033, EKB-569, GW2016, zalutumumab and/or pertuzumab/omnitarg) or a tyrosine kinase inhibitor (e.g. trastuzumab, lapatinib and/or suitnib) or a cytotoxic drug (e.g. 5-flurouracil, actinomycin D, cisplatin, cyclophosphanamide, etoposide, gemcitabine and/or vincristine). Other agents useful for combination therapy include the antimetabolites, foxuridine, cytarabine, capacetabine, gemcitabine, 6-mercaptopurine, 6-thioguanine, cladribine, fludarabine, nelarabine and/or pentostatin. In an embodiment, a dose of vinorelbine is from a single dose to a regime of less than 5 doses. Whilst the drug may be administered by any route, oral administration provides a particularly convenient route of administration. Vinorelbine is from at least 1,000 to 10,000 times more potent than methotrexate. It is proposed that a single to a dosage regime of 5 or less doses of vinorelbine or its pharmacologically active and pharmaceutically acceptable salt, solvate, tautomer, stereoisomer, derivative, biosimilar or prodrug form thereof alone or in combination with another agent is effective in selectively inducing placental cell death in the treatment of an ectopic pregnancy including unruptured ectopic pregnancy, hydatidiform molar pregnancy or choriocarcinoma or other adverse gynecological condition for which the placenta is the therapeutic target tissue. An example of another adverse condition is a placenta tumor or after hysterectomy, such as for either hydatidiform molar pregnancy or choriocarcinoma, where there is persistent disease.

It is proposed herein that the subject undergoing treatment includes a human female, in any trimester or any stage of pregnancy or pseudopregnancy or following hysterectomy. In one embodiment, a pregnant female is in her first trimester although the subject invention is not limited to any one trimester or stage of condition.

Accordingly, enabled herein is a method of treating a subject with an adverse gynecological condition, the method comprising administering to the subject an amount of vinorelbine or a pharmacologically active and pharmaceutically acceptable salt, solvate, tautomer, stereoisomer, derivative, biosimilar or prodrug from thereof effective to induce placenta cell death. For brevity, the gynecological condition is described herein as being “associated with the placenta”. This means the target tissue for treating the gynecological condition is the placenta.

A pharmaceutical preparation comprising vinorelbine or a pharmacologically active and pharmaceutically acceptable salt, solvate, tautomer, stereoisomer, derivative, biosimilar or prodrug form thereof and one or more pharmaceutically acceptable carriers, diluents and/or excipients is also contemplated herein for use in the treatment of an adverse gynecological condition associated with the placenta. The pharmaceutical preparation includes an oral formulation. In an example, the condition is an ectopic pregnancy including unruptured ectopic pregnancy, hydatidiform molar pregnancy or choriocarcinoma of the placenta or other placenta tumor or post-hysterectomy condition.

Further contemplated herein is the use of vinorelbine or a pharmacologically active and pharmaceutically acceptable salt, solvate, tautomer, stereoisomer, derivative, biosimilar or prodrug form thereof in the manufacture of a medicament to induce placenta cell death in the treatment of an adverse gynecological condition associated with the placenta. In an example, the condition is an ectopic pregnancy including an unruptured ectopic pregnancy, hydatidiform molar pregnancy, choriocarcinoma of the placenta or other placenta tumor or post-hysterectomy condition. Again, the vinorelbine may be the sole active agent or it may be used in combination with one or more other active agents.

An animal model is also enabled herein whereby an animal such as a murine animal including a mouse is injected with placenta cells (e.g. JEG3 cells), a drug such as vinorelbine (alone or in combination with other drugs) is administered and tumor volume, tumor weight and/or levels of human choriogonadotropin (hCG) measured in sacrificed animals. This enables monitoring and testing of potentially useful drugs, combination of drugs, dosages and dosage regimes. The animal model is referred to herein as a xenograft animal model. In an embodiment, the vinorelbine may be administered as the sole active agent or in combination with one or more active agents such as vinorelbine in combination with an EGFR inhibitor, a tyrosine kinase inhibitor, a cytotoxic drug or an antimetabolite. In this regard, vinorelbine may be co-formulated with the other active agent or a pharmaceutical kit is provided whereby vinorelbine and one or more other active agents are co-mixed prior to administration or co-administered separately.

The present specification further teaches a method for predicting whether an ectopic pregnancy, hydatidiform molar pregnancy, choriocarcinoma or other adverse genecology condition (e.g. another form of placenta tumor) can be successfully treated following administration of vinorelbine alone or in combination with another active agent (e.g. an EGFR inhibitor, a tyrosine kinase inhibitor, a cytotoxic agent or an antimetabolite). The predictor is the level of β-human choriogonadotropin (β-hCG). If β-hCG levels are reduced, treatment is likely to be successful.

### BRIEF DESCRIPTION OF THE FIGURES

Some figures contain color representations or entities. Color photographs are available from the Patentee upon request or from an appropriate Patent Office. A fee may be imposed if obtained from a Patent Office.

FIG. 1 is a graphical representation showing the effects of vinorelbine (VB), gefitinib (GFT) and methotrexate (MTX) or a combination thereof on cell viability.

FIGS. 2A through D are graphical representations of the effect of vinorelbine on placenta cell viability and proliferation.

FIGS. 3A through G are graphical representations of mechanisms via which vinorelbine induces cell death of placenta cells.

FIGS. 4A through F are graphical representations of the effect of vinorelbine in a mouse xenograft model.

FIGS. 5A through C are graphical representations of the effect of vinorelbine on human Fallopian tubes.

FIGS. 6A through D are graphical representations of the effect of vinorelbine on mouse fertility.

### DETAILED DESCRIPTION

Throughout this specification, unless the context requires otherwise, the word “comprise”, or variations such as “comprises” or “comprising”, will be understood to imply the inclusion of a stated element or integer or method step or group of elements or integers or method steps but not the exclusion of any other element or integer or method steps or group of elements or integers or method steps.

As used in the subject specification, the singular forms “a”, “an” and “the” include plural aspects unless the context clearly dictates otherwise. Thus, for example, reference to “a form” includes a single form, as well as two or more forms; reference to “an agent” includes a single agent, as well as two or more agents; reference to “the disclosure” includes single and multiple aspects taught by the disclosure; and so forth. Aspects taught and enabled herein are encompassed by the term “invention”. All such aspects are enabled across the width of the invention. Any variants and derivatives contemplated herein are encompassed by “forms” of the invention.

The present invention is predicated in part on the determination of the high cytotoxic sensitivity placenta cells have in the presence of the vinca alkaloid, vinorelbine. This is significant since previous therapeutic treatment of a condition such as an ectopic pregnancy involved methotrexate in a select number of patients. This can have adverse side effects including a reduction in future fertility. It can also induce collateral damage to other cell types. In the absence of a suitable pharmacological protocol other than methotrexate, surgical intervention is the only alternative which, whilst effective, is far more invasive.

Taught therein is a method of treating a subject with an adverse gynecological condition, the method comprising administering to the subject an amount of vinorelbine or a pharmacologically active and pharmaceutically acceptable salt, solvate, tautomer, stereoisomer, derivative, biosimilar or prodrug form thereof effective to induce placenta cell death. An “adverse” gynecological condition is one where placenta cells would be targeted. Hence, an “adverse gynecological condition” is one that is associated with the placenta. Examples include an ectopic pregnancy, a hydatidiform molar pregnancy, a choriocarcinoma or other placenta tumor

Reference to a “subject” means a female subject. The present protocol is effective in any trimester or stage of pregnancy or pseudopregnancy or post-hysterectomy condition or other adverse gynecological condition.

Taught herein is a method of treating a pregnant human female subject with an adverse gynecological condition associated with the placenta, the method comprising administering to the pregnant human female subject an amount of vinorelbine or a pharmacologically active and pharmaceutically acceptable salt, solvate, tautomer, stereoisomer, derivative, biosimilar or prodrug form thereof effective to induce placenta cell death.

A “gynecological condition” contemplated herein includes an ectopic pregnancy, a hydatidiform molar pregnancy and a choriocarcinoma of the placenta or other adverse condition such as another form of placenta tumor or a persistent condition following an hysterectomy. An “ectopic pregnancy” is an extrauterine pregnancy that occurs elsewhere than within the uterine cavity including the Fallopian tube, abdomen, cervix, ovary, the uterine cornua (an interstitial ectopic pregnancy) or previous cesarean scar. The term “ectopic pregnancy” includes an unruptured ectopic pregnancy as well as any extratubular ectopic pregnancy and interstitial ectopic pregnancy.

Accordingly, taught herein is a method of treating a subject with an ectopic pregnancy, the method comprising administering to the subject an amount of vinorelbine or a pharmacologically active and pharmaceutically acceptable salt, solvate, tautomer, stereoisomer, derivative, biosimilar or prodrug form thereof effective to induce placenta cell death and eliminate the ectopic pregnancy.

Further taught herein is a method of treating a subject with hydatidiform molar pregnancy or a choriocarcinoma of the placenta, the method comprising administering to the subject an amount of vinorelbine or a pharmacologically active and pharmaceutically acceptable salt, solvate, tautomer, stereoisomer, derivative, biosimilar or prodrug form thereof effective to induce placenta cell death and eliminate the molar pregnancy or choriocarcinoma.

In an embodiment, the subject is a human female subject. Accordingly, enabled herein is a method of treating a human female subject with an ectopic pregnancy, the method comprising administering to the subject an amount of vinorelbine or a pharmacologically active and pharmaceutically acceptable salt, solvate, tautomer, stereoisomer, derivative, biosimilar or prodrug form thereof effective to induce placenta cell death and eliminate the ectopic pregnancy. Further enabled is a method of treating a human female subject with a hydatidiform molar pregnancy or a choriocarcinoma of the placenta, the method comprising administering to the subject an amount of vinorelbine or a pharmacologically active and pharmaceutically acceptable salt, solvate, tautomer, stereoisomer, derivative, biosimilar or prodrug form thereof effective to induce placenta cell death and eliminate the molar pregnancy or choriocarcinoma.

Reference to “vinorelbine” encompasses pharmacologically active and pharmaceutically acceptable salts, solvates, tautomers, stereoisomers, derivatives, biosimilars and prodrug forms thereof. The vinorelbine may be used alone or in combination with another active agent such as but not limited to an epidermal growth factor receptor (EGFR) inhibitor, a tyrosine kinase inhibitor, a cytotoxic agent or an antimetabolite. An EGFR inhibitor includes gefitinib, erlotinib/tarceva, certuximab, lapatinib/tykerb, panitumumab/vectibix, theraCIMh-R3/nimotuzumab, matuzumab, MDX447, PKI166, Cl-1033, EKB-569, GW2016, zalutumumab and pertuzumab/omnitarg. In an embodiment, the EGFR inhibitor is gefitinib. A tyrosine inhibitor includes trastuzumab, lapatinib and suitnib. A cytotoxic agent includes 5-flurouracil, actinomycin D, cisplatin, cyclophosphamide, etoposide, gemcitabine and vincristine. An antimetabolite includes a folic acid antagonist, pyrimidine antagonist, purine antagonist and a deaminase inhibitor. Examples include 5-flurouracil, foxuridine, cytarabine, capacetabine, gemcitabine, 6-mercaptopurine, 6-thioguanine, cladribine, fludarabine, nelarabine and pentostatin. Reference can be made to US Patent Publication No. 2011/0110933 for more information and sources of these agents.

Accordingly, taught herein is a method of treating a subject with an ectopic pregnancy, the method comprising administering to the subject an amount of vinorelbine and an agent selected from the group consisting of an EGFR inhibitor, a tyrosine kinase inhibitor, a cytotoxic agent and an antimetabolite or a pharmacologically active and pharmaceutically acceptable salt, solvate, tautomer, stereoisomer, derivative, biosimilar or prodrug form of vinorelbine and/or the other agent effective to induce placenta cell death and eliminate the ectopic pregnancy.

Further taught herein is a method of treating a subject with a hydatidiform molar pregnancy or choriocarcinoma of the placenta, the method comprising administering to the subject an amount of vinorelbine and an agent selected from the group consisting of an EGFR inhibitor, a tyrosine kinase inhibitor, a cytotoxic agent and an antimetabolite or a pharmacologically active and pharmaceutically acceptable salt, solvate, tautomer, stereoisomer, derivative, biosimilar or prodrug form of vinorelbine and/or the other agent effective to induce placenta cell death and eliminate the molar pregnancy or choriocarcinoma.

In an embodiment, the subject is a human female subject. Accordingly, enabled herein is a method of treating a human female subject with an ectopic pregnancy, the method comprising administering to the subject an amount of vinorelbine and an agent selected from the group consisting of an EGFR inhibitor, a tyrosine kinase inhibitor, a cytotoxic agent and an antimetabolite or a pharmacologically active and pharmaceutically acceptable salt, solvate, tautomer, stereoisomer, derivative, biosimilar or prodrug form of vinorelbine and/or the other agent effective to induce placenta cell death and eliminate the ectopic pregnancy. Further enabled is a method of treating a human female subject with a hydatidiform molar pregnancy or choriocarcinoma of the placenta, the method comprising administering to the subject an amount of vinorelbine and an agent selected from the group consisting of an EGFR inhibitor, a tyrosine kinase inhibitor, a cytotoxic agent and an antimetabolite or a pharmacologically active and pharmaceutically acceptable salt, solvate, tautomer, stereoisomer, derivative, biosimilar or prodrug form of vinorelbine and/or the other agent effective to induce placenta cell death and eliminate the molar pregnancy or choriocarcinoma.

In an embodiment, taught herein is a method of treating a subject with an ectopic pregnancy, the method comprising administering to the subject an amount of vinorelbine and an EGFR inhibitor selected from the group consisting of gefitinib, erlotinib/tarceva, certuximab, lapatinib/tykerb, panitumumab/vectibix, theraCIMh-R3/nimotuzumab, matuzumab, MDX447, PKI166, Cl-1033, EKB-569, GW2016, zalutumumab and pertuzumab/omnitarg, a tyrosine inhibitor selected from the group consisting of trastuzumab, lapatinib and suitnib, a cytotoxic agent selected from the group consisting of 5-flurouracil, actinomycin D, cisplatin, cyclophosphamide, etoposide, gemcitabine and vincristine and an antimetabolite selected from the group consisting of 5-flurouracil, foxuridine, cytarabine, capacetabine, gemcitabine, 6-mercaptopurine, 6-thioguanine, cladribine, fludarabine, nelarabine and pentostatin or a pharmacologically active and pharmaceutically acceptable salt, solvate, tautomer, stereoisomer, derivative, biosimilar or prodrug form of any or all thereof effective to induce placenta cell death and eliminate the ectopic pregnancy.

Further taught herein is a method of treating a subject with a hydatidiform molar pregnancy choriocarcinoma of the placenta, the method comprising administering to the subject an amount of vinorelbine and an EGFR inhibitor selected from the group consisting of gefitinib, erlotinib/tarceva, certuximab, lapatinib/tykerb, panitumumab/vectibix, theraCIMh-R3/nimotuzumab, matuzumab, MDX447, PKI166, Cl-1033, EKB-569, GW2016, zalutumumab and pertuzumab/omnitarg, a tyrosine inhibitor selected from the group consisting of trastuzumab, lapatinib and suitnib, a cytotoxic agent selected from the group consisting of 5-flurouracil, actinomycin D, cisplatin, cyclophosphamide, etoposide, gemcitabine and vincristine and antimetabolite selected from the group consisting of 5-flurouracil, foxuridine, cytarabine, capacetabine, gemcitabine, 6-mercaptopurine, 6-thioguanine, cladribine, fludarabine, nelarabine and pentostatin or a pharmacologically active and pharmaceutically acceptable salt, solvate, tautomer, stereoisomer, derivative, biosimilar or prodrug form of any or all thereof effective to induce placenta cell death and eliminate the molar pregnancy or choriocarcinoma. The aspects above further extend to other adverse conditions such as another placenta tumor or post-hysterectomy persistent disease.

In an embodiment, the subject is a human female subject. Accordingly, enabled herein is a method of treating a human female subject with an ectopic pregnancy, the method comprising administering to the subject an amount of vinorelbine and an EGFR inhibitor selected from the group consisting of gefitinib, erlotinib/tarceva, certuximab, lapatinib/tykerb, panitumumab/vectibix, theraCIMh-R3/nimotuzumab, matuzumab, MDX447, PKI166, Cl-1033, EKB-569, GW2016, zalutumumab and pertuzumab/omnitarg, a tyrosine inhibitor selected from the group consisting of trastuzumab, lapatinib and suitnib, a cytotoxic agent selected from the group consisting of 5-flurouracil, actinomycin D, cisplatin, cyclophosphamide, etoposide, gemcitabine and vincristine and antimetabolite selected from the group consisting of 5-flurouracil, foxuridine, cytarabine, capacetabine, gemcitabine, 6-mercaptopurine, 6-thioguanine, cladribine, fludarabine, nelarabine and pentostatin or a pharmacologically active and pharmaceutically acceptable salt, solvate, tautomer, stereoisomer, derivative, biosimilar or prodrug form of any or all thereof effective to induce placenta cell death and eliminate the ectopic pregnancy. Further enabled is a method of treating a human female subject with a hydatidiform molar pregnancy or choriocarcinoma of the placenta, the method comprising administering to the subject an amount of vinorelbine and an EGFR inhibitor selected from the group consisting of gefitinib, erlotinib/tarceva, certuximab, lapatinib/tykerb, panitumumab/vectibix, theraCIMh-R3/nimotuzumab, matuzumab, MDX447, PKI166, Cl-1033, EKB-569, GW2016, zalutumumab and pertuzumab/omnitarg, a tyrosine inhibitor selected from the group consisting of trastuzumab, lapatinib and suitnib, a cytotoxic agent selected from the group consisting of 5-flurouracil, actinomycin D, cisplatin, cyclophosphamide, etoposide, gemcitabine and vincristine and antimetabolite selected from the group consisting of 5-flurouracil, foxuridine, cytarabine, capacetabine, gemcitabine, 6-mercaptopurine, 6-thioguanine, cladribine, fludarabine, nelarabine and pentostatin or a pharmacologically active and pharmaceutically acceptable salt, solvate, tautomer, stereoisomer, derivative, biosimilar or prodrug form of any or all thereof effective to induce placenta cell death and eliminate the molar pregnancy or choriocarcinoma.

In an embodiment, vinorelbine is used in combination with gefitinib. In an embodiment, taught herein is a method of treating a subject with an ectopic pregnancy, the method comprising administering to the subject an amount of vinorelbine and gefitinib or a pharmacologically active and pharmaceutically acceptable salt, solvate, tautomer, stereoisomer, derivative, biosimilar or prodrug form of vinorelbine and/or gefitinib effective to induce placenta cell death and eliminate the ectopic pregnancy.

Further taught herein is a method of treating a subject with a hydatidiform molar pregnancy or choriocarcinoma of the placenta, the method comprising administering to the subject an amount of vinorelbine and gefitinib or a pharmacologically active and pharmaceutically acceptable salt, solvate, tautomer, stereoisomer, derivative, biosimilar or prodrug form of vinorelbine and/or gefitinib effective to induce placenta cell death and eliminate the molar pregnancy or choriocarcinoma.

In an embodiment, the subject is a human female subject. Accordingly, enabled herein is a method of treating a human female subject with an ectopic pregnancy, the method comprising administering to the subject an amount of vinorelbine and gefitinib or a pharmacologically active and pharmaceutically acceptable salt, solvate, tautomer, stereoisomer, derivative, biosimilar or prodrug form of vinorelbine and/or gefitinib effective to induce placenta cell death and eliminate the ectopic pregnancy. Further enabled is a method of treating a human female subject with a hydatidiform molar pregnancy or choriocarcinoma of the placenta, the method comprising administering to the subject an amount of vinorelbine and gefitinib or a pharmacologically active and pharmaceutically acceptable salt, solvate, tautomer, stereoisomer, derivative, biosimilar or prodrug form of vinorelbine and/or gefitinib effective to induce placenta cell death and eliminate the molar pregnancy or choriocarcinoma.

It is proposed herein that vinorelbine is effective at being highly cytotoxic to placenta cells to induce cell death at dosages of from 1,000 to 10,000 times less than methotrexate. Methotrexate can be administered at from 2.5 mg to 50 mg orally per week. This may continue for weeks. In an embodiment, according to the present invention, only from 1 to 5 doses of vinorelbine are administered in an amount of from 1 mg to 200 mg per subject. In an embodiment, a single dose is administered of from 1 mg to 200 mg vinorelbine.

Reference to from “1 to 5 doses” means 1, 2, 3, 4 or 5 doses. From “1 mg to 200 mg” means 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199 or 200 mg” per dose or a fraction of a dose inbetween. Put in other terms, the dose of vinorelbine may be from 0.001 μM to 1000 μM including 0.01 μM, 0.05 μM, 0.1 μM, 1 μM, 5 μM, 10 μM and 100 μM. These dose amounts may be significantly reduced if vinorelbine is administered in combination with another active agent.

Taught herein is vinorelbine or its pharmacologically active and pharmaceutically acceptable salt, solvate, tautomer, stereoisomer, derivative, biosimilar or prodrug form or a pharmaceutical composition comprising same for use in treating a subject having an adverse gynecological condition associated with the placenta. In this regard, enabled herein is the use of vinorelbine or its pharmacologically active and pharmaceutically acceptable salt, solvate, tautomer, stereoisomer, derivative, biosimilar or prodrug form in the manufacture of a medicament for treating a subject with an adverse gynecological condition of the placenta.

In an embodiment, enabled herein is vinorelbine or its pharmacologically active and pharmaceutically acceptable salt, solvate, tautomer, stereoisomer, derivative, biosimilar or prodrug form or a pharmaceutical composition comprising same for use in treating a subject an ectopic pregnancy. In this regard, enabled herein is the use of vinorelbine or its pharmacologically active and pharmaceutically acceptable salt, solvate, tautomer, stereoisomer, derivative, biosimilar or prodrug form in the manufacture of a medicament for treating a subject with an ectopic pregnancy.

Taught herein is vinorelbine or its pharmacologically active and pharmaceutically acceptable salt, solvate, tautomer, stereoisomer, derivative, biosimilar or prodrug form or a pharmaceutical composition comprising same for use in treating a subject with a hydatidiform molar pregnancy or a choriocarcinoma. In this regard, enabled herein is the use of vinorelbine or its pharmacologically active and pharmaceutically acceptable salt, solvate, tautomer, stereoisomer, derivative, biosimilar or prodrug form in the manufacture of a medicament for treating a subject with the molar pregnancy or choriocarcinoma.

As indicated above, vinorelbine may be used alone or in combination with another agent such as an EGFR inhibitor, a tyrosine inhibitor, a cytotoxic agent or an antimetabolite.

Pharmaceutical formulations comprising an active agent such as vinorelbine are described in Remington's Pharmaceutical Sciences (1990) 18th Ed., Mack Publishing, Company. The term “pharmaceutically acceptable salt” (or solvate) refers to physiologically and pharmaceutically acceptable salts of vinorelbine and, if relevant, agents used in combination with vinorelbine.

The vinorelbine compound of the present invention or pharmaceutically active and pharmaceutically acceptable salt, solvate, tautomer, stereoisomer, derivative or biosimilar thereof may be administered as such or in the form of a suitable prodrug.

The term “prodrug” refers to a compound, which is a drug precursor and which, following administration and absorption, release the drug in vivo via some metabolic process.

Prodrugs include those that increase the bioavailability of the active agent (e.g. by allowing an orally administered compound to be more readily absorbed into the blood) or which enhance delivery of the active agent to a specific biological organ (e.g. the placenta). Thus, examples of suitable prodrugs of the substances according to the present invention include compounds modified at one or more reactive or derivatizable groups of the active agent (e.g. vinorelbine).

The agents according to the present invention including vinorelbine may be in the form of salts. The salts of the compounds of the present invention are pharmaceutically acceptable, but it will be appreciated that non-pharmaceutically acceptable salts also fall within the scope of the present invention, since these are useful as intermediates in the preparation of pharmaceutically acceptable salts.

The pharmaceutically acceptable salts include acid addition salts, base addition salts, salts of pharmaceutically acceptable esters and the salts of quaternary amines and pyridiniums. The acid addition salts are formed from a compound of the invention and a pharmaceutically acceptable inorganic or organic acid including but not limited to hydrochloric, hydrobromic, sulfuric, phosphoric, methanesulfonic, toluenesulphonic, benzenesulphonic, acetic, propionic, ascorbic, citric, malonic, fumaric, maleic, lactic, salicyclic, sulfamic or tartaric acids. The counter ion of quaternary amines and pyridiniums include chloride, bromide, iodide, sulfate, phosphate, methansulfonate, citrate, acetate, malonate, fumarate, sulfamate and tartrate. The base addition salts include but are not limited to salts such as sodium, potassium, calcium, lithium, magnesium, ammonium and alkylammonium. The salts may be made in a known manner, for example by treating the compound with an appropriate acid or base in the presence of a suitable solvent.

The compounds useful according to the present invention may be in crystalline form and/or in the form of solvates (e.g. hydrates) and it is intended that all of these forms be within the scope of the present invention. The term “solvate” is a complex of variable stoichiometry formed by a solute (in this case, a thyroid hormone agonist compound of the present invention) and a solvent. Such solvents should not interfere with the biological activity of the solute. Methods of solvation are generally known within the art.

In an aspect, the vinorelbine alone or in combination with another active agent is/are provided as a composition with a pharmaceutically acceptable carrier or diluent or excipient. Pharmaceutically acceptable vehicles and/or diluents and/or excipients include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active agent, use thereof in the therapeutic compositions is contemplated. Supplementary active ingredients can also be incorporated into the compositions.

The compounds or pharmaceutical compositions of the present invention may be administered orally, parenterally, by inhalation spray, topically, rectally, nasally, buccally, vaginally or via an implanted reservoir. The administration as used herein includes intravenous, intraperitoneal, intrauterine, percutaneous, intranasal, subcutaneous, intramuscular, intra-articular or by infusion techniques. In an embodiment, the route of administration is via oral administration.

Hence, in one aspect, the vinorelbine or pharmaceutical compositions comprising same or together with another agent are administered in any orally acceptable dosage form. Examples of suitable dosage forms include, but are not limited to, capsules, tablets, syrups, aqueous suspensions and solutions. In the case of tablets for oral use, carriers commonly used include lactose and corn starch. Lubricating agents, such as magnesium stearate, are also typically added. For oral administration in a capsule form, useful diluents include, for example, lactose. When aqueous suspensions are required for oral use, the active ingredient is combined with emulsifying and suspending agents.

Sterile injectable forms of the compositions of the present invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid or other dosage forms may also be used for the purposes of formulation.

Alternatively, vinorelbine or pharmaceutical compositions comprising same or together with another agent are administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

In another aspect, vinorelbine or pharmaceutical compositions comprising same alone or together with another active agent of this invention are administered topically. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

In another aspect, the compounds or pharmaceutical compositions are administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons and/or other conventional solubilizing or dispersing agents.

As will be readily appreciated by those skilled in the art, the route of administration and the nature of the pharmaceutically acceptable carrier will depend on the nature of the condition and the subject (e.g. human or non-human animal) to be treated. The choice of a particular carrier or delivery system, and route of administration could be readily determined by a person skilled in the art. In the preparation of any formulation containing the active agent (e.g. vinorelbine), care should be taken to ensure that the activity of the agent is not destroyed in the process and that the agent is able to reach its site of action without being destroyed. Similarly the route of administration chosen should be such that the compound reaches its site of action.

It is especially advantageous to formulate the compositions in dosage unit form for ease of administration and uniformity of dosage. Dosage unit form as used herein refers to physically discrete units suited as unitary dosages for the subjects to be treated; each unit containing a predetermined quantity of active material calculated to produce the desired therapeutic effect in association with the required pharmaceutically acceptable vehicle. The specification for the novel dosage unit forms of the present invention are dictated by and directly dependent on (a) the unique characteristics of the active agent (e.g. vinorelbine) and the particular therapeutic effect to be achieved (cytotoxicity of placenta cells); and (b) the limitations inherent in the art of compounding the active agent for the treatment of an adverse gynecological condition associated with the placenta.

Therapeutic kits or pharmaceutical packs are further contemplated herein. In an embodiment, the vinorelbine may be administered as the sole active agent or in combination with one or more active agents such as vinorelbine in combination with an EGFR inhibitor, a tyrosine kinase inhibitor, a cytotoxic drug or an antimetabolite. In this regard, vinorelbine may be co-formulated with the other active agent or a pharmaceutical kit is provided whereby vinorelbine and one or more other active agents are co-mixed prior to administration or co-administered separately. Administration of vinorelbine and another agent may be simultaneous or sequential. By “sequential” means within seconds, minutes or hours, in either order. “Simultaneous” means in the same or different formulation.

Further taught herein is an animal model. The animal model is enabled herein whereby an animal such as a murine animal including a mouse is injected with placenta cells (e.g. JEG3 cells), a drug such as vinorelbine (alone or in combination with other drugs) is administered and tumor volume, tumor weight and/or levels of human choriogonadotropin (hCG) measured in sacrificial animals. Generally, reduction in hCG levels is indicative of a positive therapeutic effect. This enables monitoring of potentially useful drugs (e.g. vinorelbine), combination of drugs and dosages. In an embodiment, the vinorelbine may be administered as the sole active agent or in combination with one or more active agents such as vinorelbine in combination with an EGFR inhibitor, a tyrosine kinase inhibitor, a cytotoxic drug or an antimetabolite. In this regard, vinorelbine may be co-formulated with the other active agent or a pharmaceutical kit is provided whereby vinorelbine and one or more other active agents are co-mixed prior to administration or co-administered separately.

In an embodiment, the animal model is a xenograft animal model such as a xenograft mouse model.

Further taught herein is a method for predicating whether an ectopic pregnancy in a subject will be successfully treated comprising:

(i) administering to the subject one or more doses of vinorelbine alone or in combination with another active agent; and

(ii) measuring the concentration of β-hCG in body fluid from the subject; wherein a reduction in the concentration of β-hCG is predictive of successful treatment of the ectopic pregnancy.

In addition, enabled herein is a method for predicating whether a hydatidiform molar pregnancy or choriocarcinoma of the placenta in a subject will be successfully treated comprising:

(i) administering to the subject one or more doses of vinorelbine alone or in combination with another active agent; and

(ii) measuring the concentration of β-hCG in body fluid from the subject;

wherein a reduction in the concentration of β-hCG is predictive of successful treatment of the molar pregnancy or choriocarcinoma of the placenta or other form of placental tumor.

### EXAMPLES

Aspects disclosed herein are further described by the following non-limiting Examples.

### Example 1

### Placenta Cell Viability of the Treatment with Vinorelbine

Vinorelbine is a vinca alkaloid which disrupts the cytoskeleton of cells. It binds β-tubulin of microtubules and disrupts microtubule dynamics. Cells cannot progress from metaphase to anaphase and this causes mitotic arrest and cell death (Jassem et al. (2001) Ann Oncol 1200:1375-1381).

### Example 2

### Minimalization of Side Effects

To minimalize side effects of vinorelbine (e.g. Jassem et al. (2001) supra), a low dose oral formulation is provided. A single dose is also trialed. Side effects are assessed by monitoring hemoglobins and neutrophil counts. If nausea, vomiting or diarrhoea is observed then these are symptomatically treated (e.g. ondansetron for nausea and loperamide for diarrhoea). Fertility is also monitored. It is proposed herein that low dose vinorelbine successfully treats an ectopic pregnancy or choriocarcinoma without adversely affecting fertility, especially at a low dose.

### Example 3

### Effects of Vinorelbine

It is proposed herein that vinorelbine is highly effective in inducing cell death in primary first trimester placenta tissue. Vinorelbine is tested on whether it activates potent apoptosis in placenta cells but not in human Fallopian tube explants. The efficacy of vinorelbine is tested in reducing human placenta tumor xenograft volume on SCID mice and mice exposed to vinorelbine are tested for fertility.

For in vitro experiments, methotrexate and vinorelbine are administered at different doses (from 10 nM to 100,000 nM). When used, gefitinib is combined with methotrexate at 8 μM. Cells/tissues are treated for 24 hours and 48 hours. Methodology for the following standard techniques are: Tissue culture (Nilsson et al. (2013) Obstet Gynecol 122(4):737-44), RNA, qPCR, immunohistochemistry (Nilsson et al. (2013) supra; Onda et al. (2015) Hypertension:10.1161/hyptertensionaha.114.04781; Kaitu'u-Lino et al. Pregnancy Hypertension: An International Journal of Women's Cardiovascular Health 2014 placenta explants (Brownfoot et al. (2015) Hypertension), FACs sorting (Nilsson et al. (2013) supra), trophoblast purification ((Nilsson et al. (2013) supra; Onda et al. (2015) supra; Kaitu'u Lino et al. (2014) supra) and western blots (Onda et al. (2015) supra).

Normally distributed data are compared using parametric tests and non-normally distributed data are compared with non-parametric tests. For in vitro studies experiments are repeated at least three times and each experiment performed at least in triplicate. Statistics are done on the median (or means) obtained from the replicate experiments, depending on data distribution.

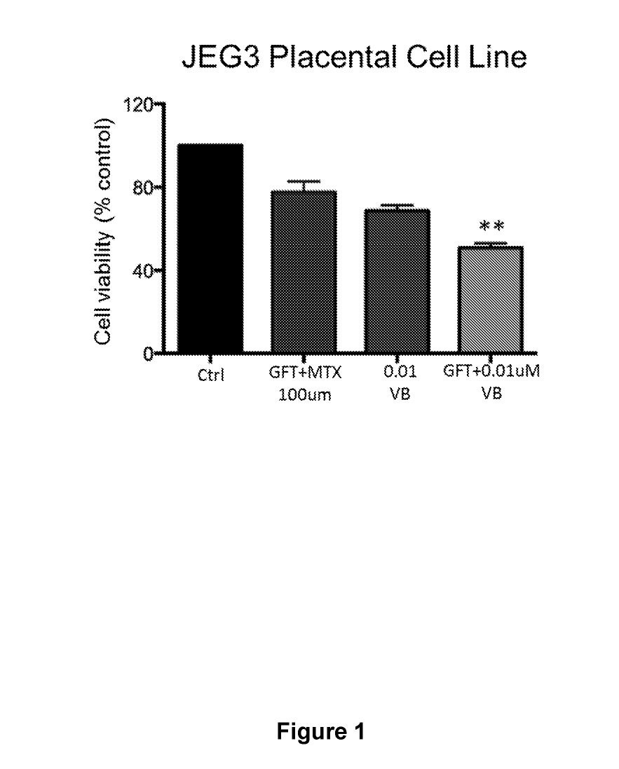

First trimester placenta tissue are obtained from women who are undergoing a termination of pregnancy. Placenta cells are purified as previously described (Nilsson et al. (2013) supra). They are treated with different doses (10-100,000 nM) of vinorelbine, methotrexate, methotrexate+gefitinib, gefitinib alone (and vehicle control). Cell viability is measured by MTS assay (see FIG. 1). To compare the relative potencies, the EC50 is calculated for the drugs (dose that induces 50% cell death).

Vinorelbine is proposed to be significantly more effective in inducing death of primary first trimester placenta cells, compared to methotrexate±gefitinib.

First trimester placenta explants are prepared and treated with the same drugs as above. After culture cells are fixed in paraformadehyde and immunohistochemistry staining performed with an M30 cytodeath antibody (detects apoptotic cell death). hCG secretion in to the cell culture media will be measured.

Vinorelbine treatment is proposed to induce more intense M30 staining of placenta explants at a lower doses, compared to methotrexate±gefitinib.

### Example 4

### Studies of Programmed Cell Death (Apoptosis)

It is useful to confirm that vinorelbine induces cell death mainly by apoptosis rather than necrosis. Apoptosis incites less of an inflammatory response that may otherwise injure local tissues. In this Example, the number of apoptotic cells after treatment with vinorelbine is assessed.

Vinorelbine is expected to induce placenta cell death mainly by apoptosis rather than necrosis. Vinorelbine is expected to induce late apoptosis.

### Example 5

### Assessing Whether Vinorelbine Causes Apoptosis on Human Fallopian Tube

It is proposed that vinorelbine will not cause significant apoptosis in the Fallopian tube given that, in contrast to the trophoblast, there are very few rapidly dividing cells. This should make it far less sensitive to vinorelbine. This is experimentally confirmed. In this Example, apoptosis is measured in Fallopian tube explants exposed to vinorelbine.

Fallopian tube cells are obtained from (non pregnant) women undergoing hysterectomy and explants prepared as previously published (Shaw et al. (2011) Am J Pathol 178(1):253-60). It is proposed to test vinorelbine, methotrexate, methotrexate+gefitinib, and gefitinib alone for 24 or 48 hours. Investigators stain the explants with M30 antibody by immunohistochemistry (measures late apoptosis) and obtain tissue lysates and perform western blot to measure the following proteins involved in apoptosis (Elmore (2007) supra): Bcl-2, Bax, Bid and Bim and quantify by densiometric analysis.

It is expected that vinorelbine will not significantly increase apoptosis in Fallopian tube explants.

### Example 6

### In Vivo Studies

Vinorelbine is proposed to be more effective than methotrexate±gefitinib in regressing placenta cells in an animal model referred to herein as a xenografted SCID mice model (Nilsson et al. (2013) supra). Mice exposed to vinorelbine remain fertile and have successful reproductive outcomes.

In this Example, in vivo evidence is obtained that vinorelbine potently regress placenta cell growth. The model is clinically relevant since those findings translate into efficacy in human trials (Skubisz et al. (2013) Obstet Gynecol 122(4):745-51; Horne et al. (2014) Hum Reprod 29(7): 1375-9).

This Example aims to compare the potency of vinorelbine vs methotrexate gefitinib in regressing placenta cell xenografts on SCID mice. Please also refer to Example 16.

An amount of 10⁶ JEG3 cells is injected subcutaneously into the flank of 5-6 week old female SCID mice (immunocompromized to accommodate the xenograft). Various treatments are administered by tail vein injection at days 6, 10, and 13 days (Nilsson et al. (2013) supra) after JEG3 inoculation.

Eight mice are included per group. This gives 80% power to detect differences in tumor size between groups of 1.3 standard deviations (calculated on the premise from previous work (Nilsson et al. (2013) supra) that the standard deviation of the JEG3 tumors is around 90 mm³).

Vinorelbine dosages are 1.25, 2.5 and 5 mg/kg animal.

From the in vivo studies of single agents detailed above, a low dose of methotrexate is selected that does not regress tumor size (the anticipated dose is 0.01 or 0.02 mg/dose) and this is combined with gefitinib at 0.5 mg/dose (previously shown adding this dose with methotrexate was super-additive (Nilsson et al. (2013) supra).

### Example 7

### Studies of Fertility

It is important to show that vinorelbine does not affect fertility. Women treated with methotrexate are asked to wait 3 months before conceiving to allow progression of 3 menstrual cycles. A shorter period of 1 month is tested in mice given their shorter estrous cycle (approximately 1 week) for the effect on fertility in mice exposed to vinorelbine.

Vinorelbine is administered by tail vein injection to wild type swiss mice. The drug (or vehicle controls) is administered by tail vein twice weekly (i.e. approximately mirroring the frequency of dosing for the xenograft studies). The middle dose that was effective in regressing tumor size (2.5 mg/kg) is to be used. After one month the mice are paired for breeding. It is proposed to sacrifice mice at about day 19 of pregnancy.

Parameters measured include 1) Number of fetuses' and implantations sites; 2) fetal crown rump length; 3) fetal weight; 4) placenta weights.

Mice exposed to vinorelbine are expected to be fertile. Compared to controls there will be the same number of fetuses', with similar crown rump lengths, fetal and placenta weights.

These preclinical studies are expected to show vinorelbine is far more potent than methotrexate in inducing placenta cell death by apoptosis and regressing placental xenografts in vivo. They will also show vinorelbine does not affect fertility, nor causes apoptosis in human Fallopian tube. A set of preclinical studies are completed to justify translating vinorelbine to clinical use.

### Example 8

### Early Phase Clinical Trial to Translate Vinorelbine into the Clinic

Vinorelbine is safe, well tolerated, and effective in resolving ectopic pregnancies. To undertake a phase I trial to determine the safety and tolerability of oral vinorelbine, the agent is administered to women with an ectopic pregnancy.

The efficacy of oral vinorelbine in resolving ectopic pregnancies vs is compared in two cohorts: 1) women presenting to a clinical service and treated with methotrexate and 2) trial participants from a phase II trial administered gefitinib and methotrexate.

Eighteen women are recruited with stable ectopic pregnancies. A phase I study dose escalation study is performed, administering 30 mg of vinorelbine (Navelbine, Pierre Fabre Ltd) on day 1 for six participants, and 60 mg for the next 12 participants. Safety and tolerability, and obtain efficacy data are examined.

In a phase I dose escalation study where the primary outcome is safety, it is not customary to perform a power calculation. However, it is noted that in a phase I trial, a cohort of six trial participants is sufficient to identify potential differences in efficacy between treatments.

Women are recruited diagnosed with a small and stable ectopic pregnancy. A small ectopic is defined by: gestational sac size <3 cm with no fetal cardiac activity on ultrasound, and pre-treatment serum hCG at presentation (day 1) between 1000-3000 IU/L. Women will not be recruited with a pre-treatment serum hCG <1000 IU/L given these are likely to successfully resolve with expectant management and do not require treatment (van Mello et al. (2013) Hum Reprod 28(1):60-7).

Stable is defined as: no clinical evidence of intra-abdominal bleeding, no pallor, guarding/rigidity on abdominal examination, stable blood pressure and heart rate, normal hemoglobin on full blood examination at day 1. An amount of 30 mg of vinorelbine is administered to the first six participants. This is then escalated to administering 60 mg of vinorelbine to a further 12 participants.

Oral vinorelbine has been administered in clinical trials once a week at either 60 mg/m² or 80 mg/m² to treat cancers. In the largest clinical trial of oral vinorelbine, neutropenia occurred in 11% of those administered 60 mg/m². In an average person of 60 kg, this equates to a weekly dose of 100 mg. Furthermore, neutropenia occurs after repeated weekly administrations. Given the preclinical studies suggest vinorelbine may be highly effective in treating ectopic pregnancy (Example 1), the drug is administered at 30, and 60 mg (the capsules are produced in 30 mg formulations). Since low doses of vinorelbine are administered, it is expected that the risk of serious neutropenia will be extremely low.

To follow up women, a protocol is used to follow up women with ectopic pregnancies medically treated with methotrexate (Anon (2008) supra). Participants are monitored on an outpatient basis.

Safety and tolerability is assessed for evidence that the ectopic pregnancy has ruptured, and to record side effects. Gastrointestinal (nausea and diarrhoea) symptoms are noted and treat symptomatically if required (ondansetron for vomiting and loperamide for diarrhoea). The inventors perform the following investigations to screen for toxicity: liver function tests, urea and electrolytes (monitors renal function), and full blood examination on days 1, 4 and 7, then weekly.

Assessment of treatment response: Serum hCG is measured on day 4 and 7. A drop of 15% of serum hCG between these time-points will be taken as evidence that the treatment is working (which is the same as widely used protocols to monitor response after treatment with methotrexate). Serum hCG is tracked weekly until it reaches zero.

Those that have not had a serum hCG level that has fallen 15% from day 4 to day 7 are deemed as treatment failure.

A decrease of serum hCG to zero without need for salvage treatment with either surgery or methotrexate is evidence of cure. The patients are contacted on a six monthly basis to record both the return of normal menstrual period cycles, and to identify those who go on to have subsequent pregnancies. Pregnancy outcomes are obtained.

Descriptive statistics are used to describe clinical details of adverse outcomes including side effects (on history and examination) and any abnormalities in laboratory indices.

Given this is a phase I dose escalation trial, the primary outcome is safety and tolerability. However, preliminary data are obtained on efficacy by performing a similar analysis as phase I trial of gefitinib and methotrexate (Skubisz et al. (2013) supra). The results obtained are compared with vinorelbine (n=18) with the following cohorts: 1) methotrexate: contemporaneous cohort who presented to clinical services with a pretreatment serum hCG 1000-3000 IU/L and were successfully treated with methotrexate; 2) combination gefitinib and methotrexate: the phase II trial of gefitinib and methotrexate (16 participants with a pre-treatment serum hCG 1000-3000 IU/L, and were successfully treated).

Compared are 1) the time to resolution of the ectopic pregnancy (in days) and 2) serum hCG levels at days 1, 4 and 7. Also compared are the same outcomes (time to resolution and serum hCG levels at days 1, 4 and 7) between the six participants administered 30 mg of vinorelbine vs 12 administered 60 mg of vinorelbine. Previous phase I data suggest if a treatment effect is large, a cohort of six participants is sufficient to identify differences in either of these treatment outcomes (Skubisz et al. (2013) supra).

Statistics are used to compare these efficacy outcomes. Categorical values (i.e. time to resolution) are compared using chi-squared test and continuous data are compared using the appropriate parametric (T-test if normally distributed) or non-parametric test (Mann-Whitney U).

Oral vinorelbine is expected to be safe and well tolerated. Vinorelbine is expected to resolve ectopic pregnancies significantly faster than methotrexate±gefitinib. This phase I trial provides data justifying definitive phase II-III trials. The results are expected to lead to the use of single dose oral vinorelbine as the standard of care to treat ectopic pregnancy.

It is proposed herein that stable ectopic pregnancies is effectively treated with a single tablet, taken once. Furthermore, treatment can be monitored by measuring hCG. A reduction in hCG levels is indicative of successful treatment.

### Example 9

### Combination of Vinorelbine and Gefitinib

FIG. 1 shows the effects of a combination of gefitinib (GFT) and vinorelbine (VB) compared to GFT and methotrexate (MTX). VB doses were 10,000 times lower than MXT. Data show that VB+GFT is more potent at inducing cell death at a dose that is 10,000 times lower than MTX.

### Example 10

### Effect of Vinorelbine on Placenta Cell Viability and Proliferation

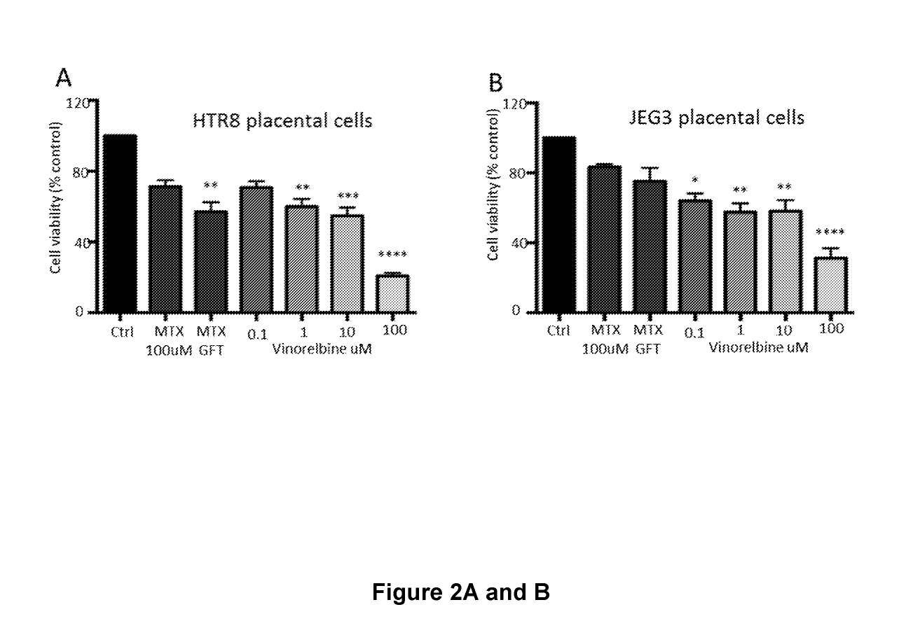

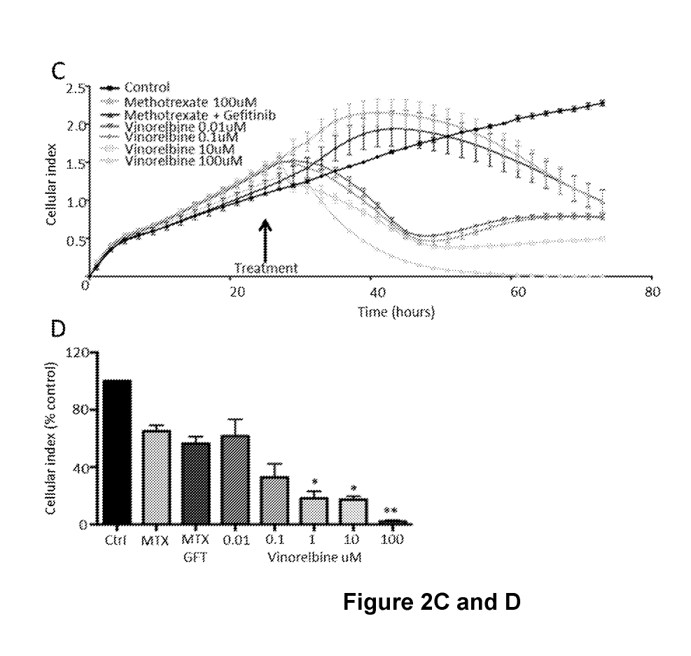

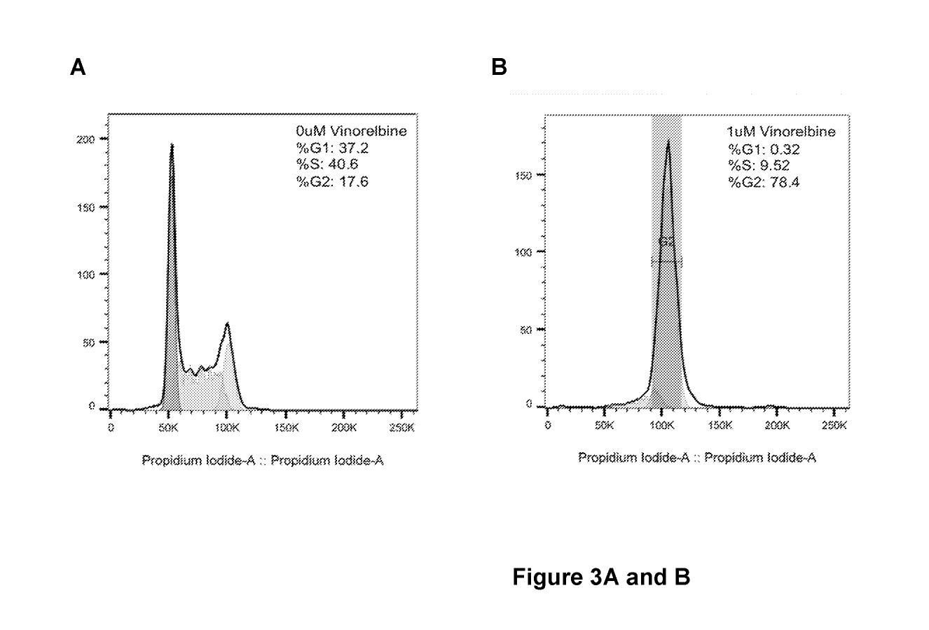

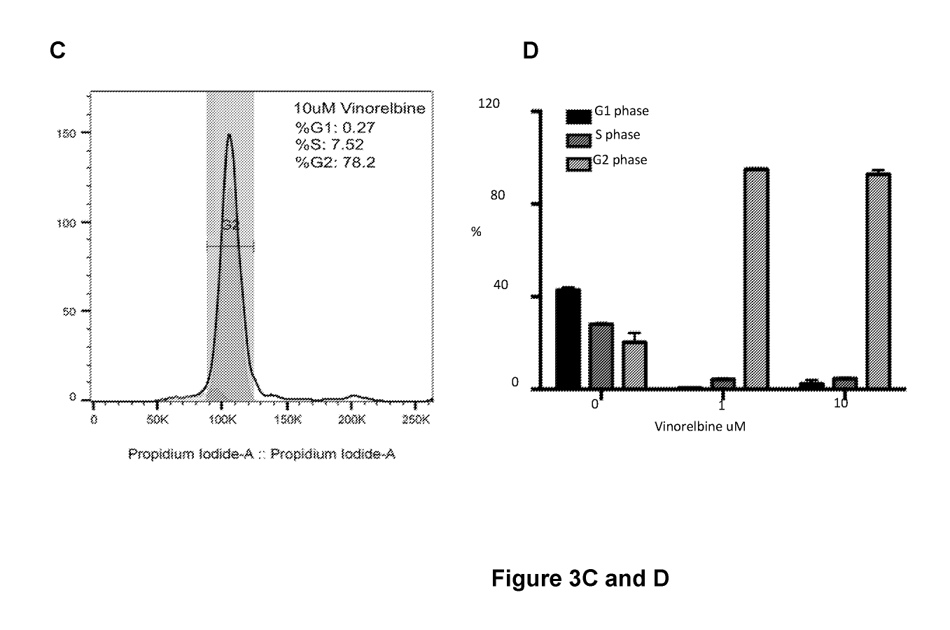

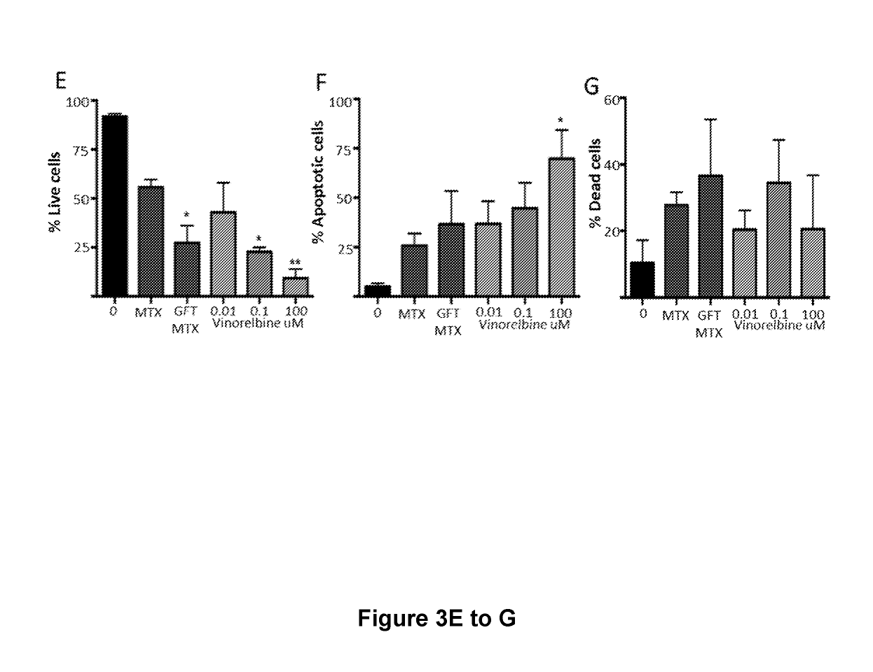

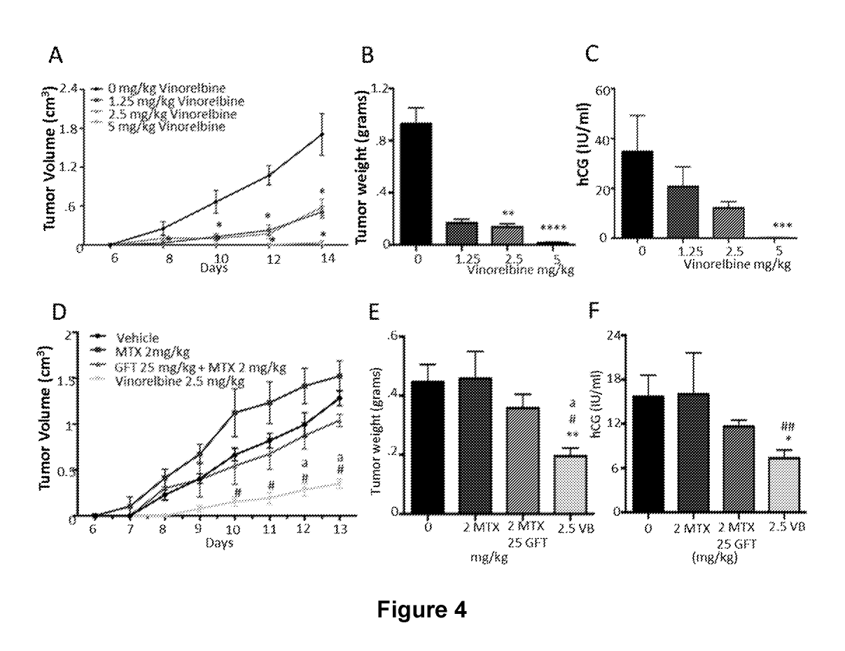

The ability for vinorelbine to reduce placenta cell viability was assessed in two placenta cell lines (A;HTR8, B; JEG3) using a MTS cell viability assay. Results are shown in FIGS. 2A through D. Treatment with vinorelbine induced a significant dose dependent reduction in cell viability in both cell types, which was more significant than either methotrexate alone (MTX) or methotrexate plus gefitinib (MTX+GFT).

The effect of vinorelbine on placenta cell (HTR8) proliferation was assessed using xCELLigence (C). Vinorelbine reduced HTR8 proliferation in a dose-dependent manner and appeared to do so earlier than methotrexate or methotrexate plus gefitinib. When data are analyzed at 48 h post plating (D) it was found that proliferation was reduced at 1, 10 and 100 μM of vinorelbine, but no significant effect of methotrexate for methotrexate+gefitinib compared to control. Data expressed as mean±SEM. *p<0.05, **p<0.01, ***p<0.001, ****p<0.0001 vs. control. Together these data show that vinorelbine is more potent at reducing placenta cell viability and inhibiting proliferation than methotrexate alone or combination methotrexate plus gefitinib.

### Example 11

### Mechanisms Via which Vinorelbine Induces Cell Death of Placenta Cells

The effect of Vinorelbine on cell cycle progression in HTR8 cells was assessed using FACs analysis (FIGS. 8A through G). Vinorelbine at 1 μM (B) and 10 μM (C) inhibits cell cycle progression, with cells arrested in the G2 phase of mitosis. The proportion of cells in G1, S and G2 is graphically represented in n=3 replicates (D). Compared to control (0 vinorelbine), there are significantly more cells halted at G2 in the 1 μM and 10 μM vinorelbine treated groups.

To further assess the effects of vinorelbine in HTR8s, flow cytometry was undertaken to determine whether vinorelbine enhances cells death and apoptosis. Treating with combination methotrexate+gefitinib and vinorelbine alone significantly reduced the percentage of live HTR8 cells (E). Vinorelbine alone significantly increased the percentage of apoptotic HTR8 cells (F) assessed via Annexin V expression. No significant changes was found in the percentage of dead cells (G). Data expressed as mean±SEM. *p<0.05, **p<0.01. Together these data demonstrate that vinorelbine's mechanism for reducing cell viability and cellular proliferation in placenta cells occurs as a result of impairment of the cell cycle at G2 and via increased apoptosis.

### Example 12

### Effect of Vinorelbine in a Mouse Xenograft Model

Given there are no good animal models of ectopic pregnancy, a mouse xenograft model was previously devised, where placenta cells (JEG3) are grafted subcutaneously on to the flank of immuno-compromised mice and a placenta tumor allowed to form (n=6-8 per group). Results are shown in FIGS. 9A through F. The effect of increasing doses of vinorelbine (A-C) was initially assessed. Intravenously administered vinorelbine dose dependently reduced tumor volume over time (A). This was reflected by significantly reduced tumor weight (B) and serum human chorionic gonadotropin (hCG) levels (C). Vinorelbine was compared with methotrexate alone and combination methotrexate plus gefitinib. Compared to methotrexate alone and methotrexate+gefitinib, vinorelbine significantly reduced tumor volume over time (D), tumor weight (E) and serum HCG levels (F). Data expressed as mean±SEM. *p<0.05, **p<0.01, ***p<0.001 vs. vehicle treated. a p<0.05 vs. Gefitinib+Methotrexate. # p<0.5. ## p<0.01 vs. Methotrexate. Together these data demonstrate that vinorelbine can effectively inhibit placenta cell growth in vivo.

### Example 13

### Effect of Vinorelbine on Human Fallopian Tubes

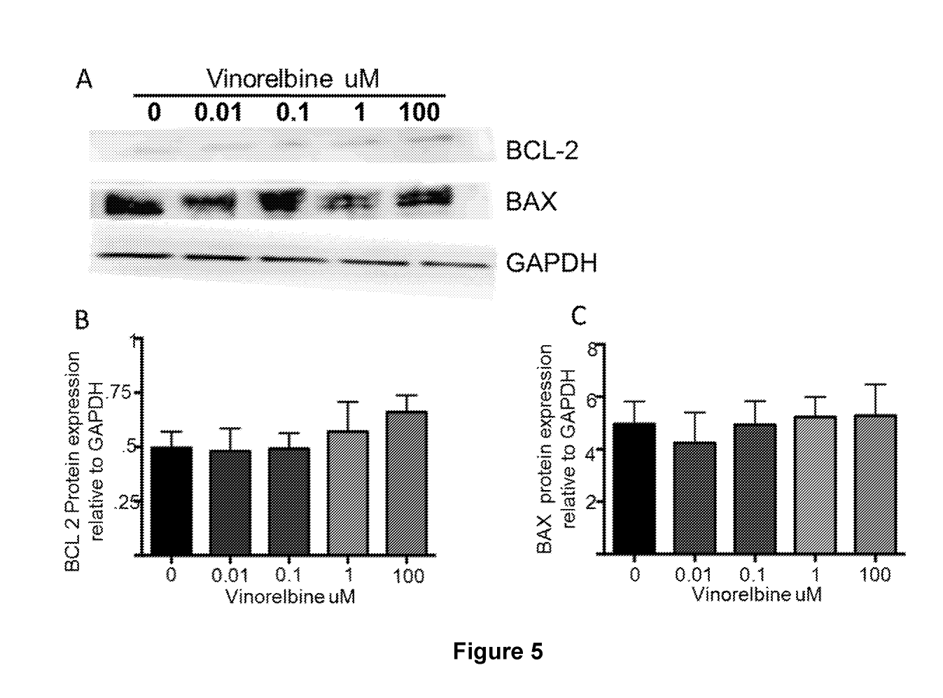

For preservation of future fertility, it is important that vinorelbine does not cause and damage to the human Fallopian tube. Human Fallopian tubes were obtained at hysterectomy (from women of child-bearing age). Human Fallopian tubes were treated ex vivo with vinorelbine and the effect on apoptotic markers BCL 2 and BAX protein expression determined via western blot (FIG. 5A). Densitometric analysis of western blots performed on n=3 tubes (From 3 separate patients) confirmed no effect (FIGS. 5B, C). It is proposed to collect n=8 Fallopian tubes in total in order to assess other apoptotic markers, including caspase-3 and PARP. A cell death stain is also be performed.

Preliminary data suggest that vinorelbine does not upregulate markers of apoptosis in human Fallopian tubes.

### Example 14

### Effect of Vinorelbine on Mouse Fertility

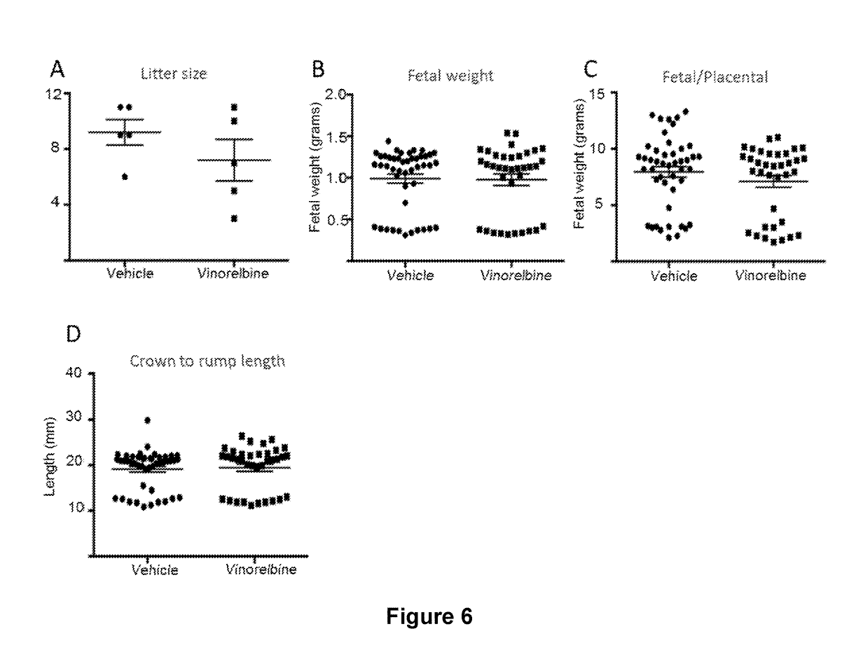

To determine whether vinorelbine would affect future fertility, breeding experiments in mice were undertaken (FIG. 6). Intravenous administration of the highest dose of 5 mg/kg of vinorelbine to female mice (n=5-6 per a group) 4 weeks prior to breeding did not alter litter size (A), fetal weight (B), fetal/placenta ratio (C) or crown to rump length (D).

In the clinic, females are advised to wait a minimum of three months post methotrexate treatment for ectopic pregnancy before attempting to conceive again. For future studies it is planned to repeat this experiment with 3 doses of 2.5 mg/kg vinorelbine (as performed in xenograft experiments) treatment before breeding (n=8 mice/group).

Those skilled in the art will appreciate that the disclosure described herein is susceptible to variations and modifications other than those specifically described. It is to be understood that the disclosure contemplates all such variations and modifications. The disclosure also enables all of the steps, features, compositions and compounds referred to or indicated in this specification, individually or collectively, and any and all combinations of any two or more of the steps or features or compositions or compounds.

### BIBLIOGRAPHY

Anon (1995) Morbidity Mortality Weekly Report 44:46-48
Anon (2008) Obstet Gynecol 111(6):1479-1485
Brownfoot et al. (2015) Hypertension
Cecchno et al. (2014) Arch Glynecol Obstet 290(3):417-423
Elmore (2007) Toxiol Pathol 35(4):495-516
Farquhar (2005) Lancet 366 (9485):583-591
Horne et al. (2014) Hum Reprod 29(7):1375-9
Jassem et al. (2001) Ann Oncol 12(10):1375-1381
Kaitu'u-Linao et al. (2013) Endocrinology 154(2):911-9
Kaitu'u-Lino et al. Pregnancy Hypertension: An International Journal of Women's Cardiovascular Health 2014
Kirk et al. (2014) Hum Reprod Update 20(2):250-261
Mol et al. (2008) Hum Reprod Update 14(4):309-319
Nilsson et al. (2013) Obstet Gynecol 122(4):737-44
Onda et al. (2015) Hypertension:10.1161/hyptertensionaha.114.04781
Pisarska and Carson (1999) Clin Obstet Gynecol 42(1):2-8
Remington's Pharmaceutical Sciences (1990) 18th Ed., Mack Publishing, Company
Shaw et al. (2011) Am J Pathol 178(1):253-60
Skubisz et al. (2013) Obstet Gynecol 122(4):745-51
van Mello et al. (2013) Hum Reprod 28(1):60-7

1. A method of treating a human female subject with an adverse gynecological condition associated with the placenta, said method comprising administering to said subject an amount of vinorelbine or a pharmacologically active and pharmaceutically acceptable salt, solvate, tautomer, stereoisomer, derivative, biosimilar or prodrug form thereof effective to induce placenta cell death.

2. The method of claim 1 wherein the human female subject has an ectopic pregnancy.

3. The method of claim 2 wherein the human female subject has an unruptured ectopic pregnancy, extratubular ectopic pregnancy or an interstitial ectopic pregnancy.

4. The method of claim 1 wherein the subject has choriocarcinoma of the placenta or another form of placenta tumor.

5. The method of claim 1 wherein the subject has a hydatidiform molar pregnancy.

6. The method of claim 1 further comprising administering vinorelbine, simultaneously or sequentially, with another active agent.

7. The method of claim 6 wherein the other active agent is selected from the group consisting of an epidermal growth factor receptor (EGFR) inhibitor, a tyrosine kinase inhibitor, a cytotoxic agent and an antimetabolite.

8. The method of claim 7 wherein the EGFR is selected from the group consisting of gefitinib, erlotinib/tarceva, certuximab, lapatinib/tykerb, panitumumab/vectibix, theraCIMh-R3/nimotuzumab, matuzumab, MDX447, PKI166, Cl-1033, EKB-569, GW2016, zalutumumab and pertuzumab/omnitarg.

9. The method of claim 7 wherein the tyrosine kinase inhibitor is selected from the group consisting of trastuzumab, lapatinib and suitnib.

10. The method of claim 7 wherein the cytotoxic agent is selected from the group consisting of 5-flurouracil, actinomycin D, cisplatin, cyclophosphamide, etoposide, gemcitabine and vincristine.

11. The method of claim 7 wherein the antimetabolite is selected from the group consisting of 5-flurouracil, foxuridine, cytarabine, capacetabine, gemcitabine, 6-mercaptopurine, 6-thioguanine, cladribine, fludarabine, nelarabine and pentostatin.

12. The method of claim 7 wherein the EGFR inhibitor is gefitinib or a pharmacologically active and pharmaceutically acceptable salt, solvate, tautomer, stereoisomer, derivative, biosimilar or prodrug form thereof.

13. The method of claim 1, wherein the vinorelbine is given in an oral dosage form.

14. The method of claim 13 wherein from 1 to 5 oral doses of vinorelbine is administered.

15. The method of claim 13 wherein from 1 to 200 mg of vinorelbine is administered orally.

16. The method of claim 15 wherein from 10 to 100 mg of vinorelbine is administered orally.

17. The method of claim 6, wherein vinorelbine is administered with a separate or combined oral dosage form of another active agent.

18.-31. (canceled)

32. The method according to claim 1, wherein the method is a therapeutic protocol to treat a female human subject with:
(a) an ectopic pregnancy,
(b) choriocarcinoma of the placenta or other form of placenta tumor, or
(c) a hydatidiform molar pregnancy,
said protocol comprising administering to the female human subject of from 1 to 5 doses of vinorelbine at from 1 mg to 200 mg per dose.

33. (canceled)

34. (canceled)

35. The therapeutic protocol of claim 32, wherein said protocol comprises administering a single dose of vinorelbine at from 1 mg to 200 mg per dose.

36. A method for predicating whether an adverse gynecological condition associated with a placenta in a subject will be successfully treated comprising:
(i) administering to the subject one or more doses of vinorelbine alone or in combination with another active agent; and
(ii) measuring the concentration of β-hCG in body fluid from the subject;
wherein a reduction in the concentration of β-hCG is predictive of successful treatment of the adverse gynecological condition associated with a placenta,
wherein the adverse gynecological condition associated with a placenta is selected from the group consisting of an ectopic pregnancy, a choriocarcinoma of the placenta or other form of placenta tumor, and a hydatidiform molar pregnancy.

37. (canceled)

38. (canceled)
